(19) United States
(12) Patent Application Publication
(10) Pub. No.: US 2024/0230605 A1
(43) Pub. Date: Jul. 11, 2024

(54) IMPROVEMENTS TO PEAK INTEGRATION BY INTEGRATION PARAMETER ITERATION

(71) Applicant: DH Technologies Development Pte. Ltd., Singapore (SG)

(72) Inventors: Lyle L. BURTON, Woodbridge (CA); Stephen TATE, Barrie (CA)

(73) Assignee: DH Technologies Development Pte. Ltd., Singapore (SG)

(21) Appl. No.: 18/558,592

(22) PCT Filed: May 4, 2022
(86) PCT No.: PCT/IB2022/054136

§ 371 Date: Nov. 2, 2023

Related U.S. Application Data
(60) Provisional application No. 63/184,665, filed on May 5, 2021.

(51) Int. Cl. G01N 30/86

(52) U.S. Cl. CPC G01N 30/8634; G01N 30/8606; G01N 30/8693

(57) ABSTRACT

Methods and systems for improving peak integration in mass spectrometry. A method may include accessing an ion data series; generating a set of prospective peak integrations for a target peak in the ion data series; providing, as input to a trained machine learning model, at least one peak characteristic for each prospective peak integration in the set of prospective peak integrations; processing the provided input, by the trained machine learning model, to generate an output from the trained machine learning model; based on the output, generating a ranking of one or more of the prospective peak integrations; and based on one of the prospective peak integrations, generating an ion amount represented by the target peak.

### CROSS REFERENCE TO RELATED APPLICATION

The application is being filed on May 4, 2022, as a PCT International Patent Application that claims priority to and the benefit of U.S. Provisional Application No. 63/184,665 filed on May 5, 2021, which application is hereby incorporated herein by reference.

### BACKGROUND

Mass spectrometry (MS) is an analytical technique for detection and quantitation of chemical compounds based on the analysis of m/z values of ions formed from those compounds. MS involves ionization of one or more compounds of interest from a sample, producing precursor ions, and mass analysis of the precursor ions. Tandem mass spectrometry or mass spectrometry/mass spectrometry (MS/MS) involves ionization of one or more compounds of interest from a sample, selection of one or more precursor ions of the one or more compounds, fragmentation of the one or more precursor ions into product ions, and mass analysis of the product ions.

Mass spectrometers are often coupled with chromatography or other separation systems in order to identify and characterize eluting compounds of interest from a sample. In such a coupled system, the compounds in the eluting solvent are ionized and a series of mass spectra are obtained at specified time intervals. These times range from, for example, 1 second to 100 minutes or greater. Intensity values derived from the series of mass spectra form a chromatogram. For example, the sum of all intensities generates a Total Ion Chromatogram (TIC) and the intensity of one mass value generates an extracted ion chromatogram (XIC).

Whether chromatography systems are used or not, a signal or data series representing the ions counted by the mass spectrometry system is generated. A series of peaks are formed by the ion signal or ion data series. The peaks found in the ion data series may be used to quantify amounts of analytes within the sample at particular mass-to-charge ratios. For example, peaks found in chromatograms are used to identify or characterize a known peptide or compound in the sample because they elute at known times called retention times. More particularly, the retention times of peaks and/or the area of peaks are used to identify or characterize (quantify) a known peptide or compound in the sample.

In traditional separation coupled mass spectrometry systems, a precursor ion of a known compound is selected for analysis. An MS/MS scan is then performed at each interval of the separation for a mass range that includes the precursor ion. The intensity of the product ions found in each MS/MS scan is collected over time and analyzed as a collection of spectra, or an XIC, for example. Both MS and MS/MS can provide qualitative and quantitative information.

The measured precursor or product ion spectrum can be used to identify a molecule of interest. The intensities of precursor ions and product ions can also be used to quantitate the amount of the compound present in a sample.

As described above, mass spectrometers are often coupled with separation systems or devices in order to identify and characterize eluting compounds of interest from a sample. Such separation devices can include, but are not limited to, liquid chromatography (LC) devices, gas chromatography devices, capillary electrophoresis devices, or ion mobility devices. LC devices are commonly used in conjunction with mass spectrometers to quantify the amount of a compound of interest in a sample.

### SUMMARY

In an aspect, the technology relates to a method for improving mass spectrometry system measurement. The method includes accessing an ion data series for an ion count rate generated from ions detected by a detector of a mass spectrometry system; generating a set of prospective peak integrations for a target peak in the ion data series, wherein each prospective peak integration in the set of prospective peak integrations is generated based on a different set of peak integration parameters, and each prospective peak integration is characterized by at least one peak characteristic; providing, as input to a trained machine learning model, the at least one peak characteristic for each prospective peak integration in the set of prospective peak integrations; processing the provided input, by the trained machine learning model, to generate an output from the trained machine learning model; based on the output, generating a ranking of one or more of the prospective peak integrations; and based on one of the prospective peak integrations, generating an ion amount represented by the target peak.

In an example, the method further includes causing a display of one or more of the prospective peak integrations based on the ranking; receiving a selection of one of the displayed prospective peak integrations; and wherein generating the ion amount is based on the selected prospective peak integration. In another example, the peak integration parameters include at least one of a smoothing parameter, an expected-time parameter, a filtering parameter, a baseline parameter, or a peak-splitting parameter. In yet another example, the at least peak characteristic includes at least one of: an integrated area, peak height, peak start time, peak end time, center time, peak width, and peak smoothness. In still another example, each prospective peak integration in the set of prospective peak integrations includes at least one respective peak quality metric, and the respective peak quality metrics are also included as input into the trained machine learning model. In still yet another example, one or more of the peak integration parameters are also included as input to the trained machine learning model.

In another example, the set of prospective peak integrations includes at least 50 prospective peak integrations. In a further example, the trained machine learning model is one a neural network, a support vector machine, a K-nearest neighbors algorithm, a hidden Markov model, or a random forest. In still another example, the ion data series is part of a chromatogram. In yet another example, data points within the data series indicate an ion count rate and sampling interval time.

In another aspect, the technology relates to a system for improving mass spectrometry system measurement. The system includes at least one processor; and memory storing instructions that, when executed by the at least one processor, cause the system to perform operations. The operations include access an ion data series for an ion count rate generated from ions detected by a detector of a mass spectrometry system; generate a set of prospective peak integrations for a target peak in the ion data series, wherein each prospective peak integration in the set of prospective peak integrations is generated based on a different set of peak integration parameters, and each prospective peak integration is characterized by at least one peak characteristic; provide, as input to a trained machine learning model, the at least one peak characteristic for each prospective peak integration in the set of prospective peak integrations; process the provided input, by the trained machine learning model, to generate an output from the trained machine learning model; based on the output, generate a ranking of one or more of the prospective peak integrations; and based on one of the prospective peak integrations, generate an ion amount represented by the target peak.

In an example, the system further includes a display and an input device, and the operations further include display, on the display, of the prospective peak integrations based on the ranking; receive, via the input device, one of the displayed prospective peak integrations; and wherein generating the ion amount is based on the selected prospective peak integration. In a further example, the peak integration parameters include at least one of a smoothing parameter, an expected-time parameter, a filtering parameter, a baseline parameter, or a peak-splitting parameter. In another example, the at least one peak characteristic includes at least two of: an integrated area, peak height, peak start time, peak end time, center time, peak width, and peak smoothness. In still another example, each prospective peak integration in the set of prospective peak integrations includes at least one respective peak quality metric, and the respective peak quality metrics are also included as input into the trained machine learning model.

In another aspect, the technology relates to a method for improving mass spectrometry system measurement. The method includes accessing an ion data series for an ion count rate generated from ions detected by a detector of a mass spectrometry system; generating, according to first peak integration parameters, a first prospective peak integration for an identified peak in the ion data series, wherein the first prospective peak integration is characterized by first peak characteristics; generating, according to second peak integration parameters, a second prospective peak integration for the identified peak in the ion data series, wherein the second prospective peak integration is characterized by second peak characteristics; providing, as input to a trained machine learning model: the first peak characteristics; and the second peak characteristics; processing the provided input, by the trained machine learning model, to generate an output from the trained machine learning model; based on the output, generating a ranking of the first prospective peak integration and second prospective peak integration; and based on at least one of the first prospective peak integration or the second prospective peak integration, generating an ion amount represented by the peak.

In an example, the method further comprises causing the display of at least one of the first prospective peak integration or the second prospective peak integration based on the ranking; receiving a selection of one of the first prospective peak integration or the second prospective peak integration; and wherein generating the ion amount is based on the selected prospective peak integration. In another example, the peak integration parameters include at least one of a smoothing parameter, an expected-time parameter, a filtering parameter, a baseline parameter, or a peak-splitting parameter. In still another example, the peak characteristics include at least two of: an integrated area, peak height, peak start time, peak end time, center time, peak width, and peak smoothness. In yet another example, the trained machine learning model is one a neural network, a support vector machine, a K-nearest neighbors algorithm, a hidden Markov model, or a random forest. In a further example, the first prospective peak integration has a first peak quality metric, the second prospective peak integration has a second peak quality metric, and the input to the trained machine learning model further includes the first peak quality metric and the second peak quality metric.

In another aspect, the technology relates to a method for improving mass spectrometry system measurement. The method includes accessing an ion data series for an ion count rate generated from ions detected by a detector of a mass spectrometry system; identifying peaks corresponding to samples having known analyte concentrations; for each identified peak, generating a set of prospective peak integrations for the identified peak in the ion data series, wherein each of the prospective peak integrations is generated according to different peak integration parameters; for multiple combinations of the generated sets of prospective peak integrations, fitting a curve to the prospective peak integrations in the respective combination; identifying a subset of the generated prospective peak integrations based on at least one of a curve fit or accuracy score of the respective fitted curve; and generating an ion amount for a sample having an unknown concentration based on peak integration parameters of one of the prospective peak integrations in the identified subset of the prospective peak integrations.

In an example, each prospective peak integration is characterized by peak characteristics. In another example, the method further includes providing, as input to a trained machine learning model, the peak characteristics for the subset of prospective peak integrations; processing the provided input, by the trained machine learning model, to generate an output from the trained machine learning model; and based on the output, generating a ranking of one or more of the prospective peak integrations in the subset of prospective peak integrations. In yet another example, the method further includes causing a display of one or more of the prospective peak integrations in the subset of prospective peak integrations based on the ranking; receiving a selection of one of the displayed prospective peak integrations; and wherein generating the ion amount is based on the selected prospective peak integration.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

Non-limiting and non-exhaustive examples are described with reference to the following figures.

FIG. 1 depicts an example system for performing mass spectrometry.

FIG. 2 is an example user interface showing the peak-finding or peak integration parameters used by the peak finding algorithm to integrate a peak.

FIG. 3 depicts an example chromatogram explaining the use of a peak-integration parameter.

FIG. 4 depicts another chromatogram explaining the use of another peak-integration parameter.

FIG. 5 depicts a portion of a chromatogram explaining the use of another peak-integration parameter.

FIG. 6 depicts three example prospective peak integrations generated from three different sets of integrations parameters.

FIG. 7 depicts an example system for predicting top prospective peak integrations using a trained machine learning (ML) model.

FIG. 8A depicts example plots for standardized results using different prospective peak integrations.

FIG. 8B depicts the plots of FIG. 8A combined into a single plot.

FIG. 9 depicts an example method for improving mass spectrometry measurements.

FIG. 10 depicts another example method for improving mass spectrometry measurements.

### DETAILED DESCRIPTION

As discussed briefly above, the output of a mass spectrometry system may be an ion data series with data points representative of ion counts. The ion data series may be represented in a variety of manners, one of which is a chromatogram in examples where a chromatography device is used. Chromatograms are essentially a collection of ion counts or intensities that are a function of time. Chromatograms are often used to determine the quantity of a particular compound that is present in a sample. In order to quantify or quantitate a compound, either a precursor ion or product ion peak in a chromatogram is integrated. Integration of a peak generally refers to finding the area under the peak in the chromatogram. The accuracy of the peak integrations is important for the ultimate accuracy of the quantification of the result. The peak integration may be based on a set of parameters that are variable, and one set of parameters may include a more accurate peak integration than another set of parameters. Identification and selection of the correct or best set of parameters for peak integration, however, continues to be a problem and a challenge. In some cases, the process is performed manually, leading to substantial time consumption as well as inconsistency and subjectivity in the results. Additional discussion regarding the peak integration problem is provided in International Publication Number WO 2020/250158, which is incorporated herein by reference in its entirety.

The present technology helps address the peak-integration problem by automatically determining the best set or sets of peak-integration parameters, which ultimately may lead to an improved, more accurate, and more consistent mass spectrometry system. For example, the present technology generates sets of prospective peak integrations by changing or iterating over different peak integration parameters. The prospective peak integrations may be defined by peak characteristics, such as an integrated area, peak height, peak start time, peak end time, center time, peak width, and peak smoothness. The prospective peak integrations may also have associated peak quality metrics that indicate potential quality or accuracy of the prospective peak integrations. One or more of the peak-integration parameters, the peak characteristics, and/or the peak quality metrics may be provided as input into a trained machine learning model. The trained machine learning model then processes the input to generate an output indicating the top set prospective peak integrations. In some examples, the top set of prospective peak integrations may be presented to a user for selection of one or the prospective peak integrations for the analysis of the sample. In other examples, the present technology may automatically use the top-ranked prospective peak integration for the analysis of the sample.

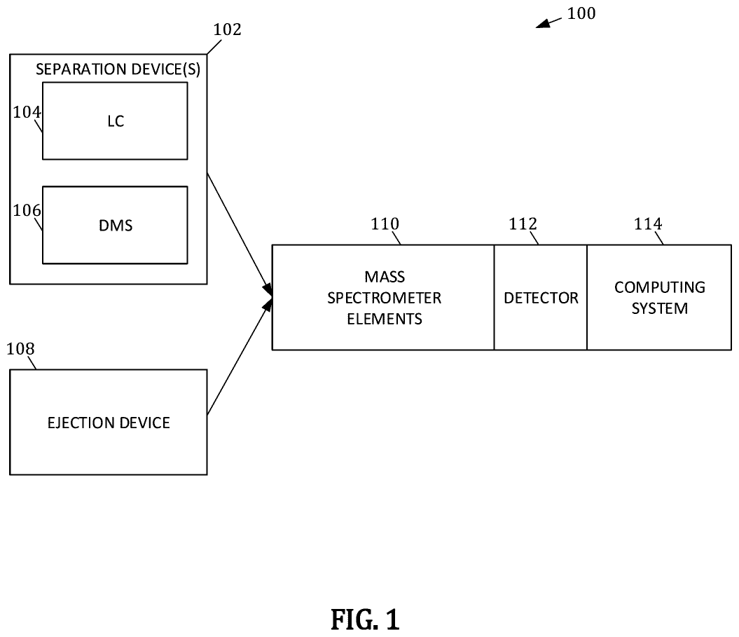

FIG. 1 depicts an example mass analysis system 100 for performing mass spectrometry techniques. The example system 100 may include one or more separation devices 102 that separate a sample such that different analytes of the sample may be analyzed as the sample passes through or elutes from the separation devices 102. For example, the system 100 may include a liquid chromatography (LC) device and/or a differential mobility separation (DMS) device 106. An LC device may include two separate devices, such as a high-performance liquid chromatography (HPLC) device and a direct infusion or injection device. In an HPLC device, one of two solvents or is selected using valve. Solvents are moved to the valve using pumps. The sample is mixed with the selected solvent using mixer, and the resulting mixture is sent through a liquid chromatography (LC) column. In the direct infusion or injection device, a sample may already be mixed with a solvent in fluidic pump.

Other types of separation devices 102 may also be utilized, such as a gas chromatography device or a capillary electrophoresis device, among others. Instead of, or in addition, to the separation devices 102, the system 100 may include an ejection device 108. The ejection device may be an acoustic ejection device that acoustically ejects droplets from the sample for analysis.

The separation device 102 and/or the ejection device 108 introduces a portion of the sample into a series of mass spectrometer elements 110 that may be a mass spectrometer. For instance, the mass spectrometer may be any type of mass spectrometer, including a quadrupole mass spectrometer, a quadrupole or triple quadrupole (QqQ), an ion trap, an orbitrap, a time-of-flight (TOF) mass spectrometer, or a Fourier transform (FT) mass spectrometer. The mass spectrometer or mass spectrometer elements 110 may also include an ionization device for ionizing the portions of the sample to generate ions that are accelerated through the mass analysis components of the mass spectrometer.

The system 100 includes a detector that may be part of the mass spectrometer. The detector may include an electron multiplier detector that may include analog-to-digital conversion (ADC) circuitry or an image-charge detector. An ADC detector detects impacts of ions on the detector to generate a count or intensity of ions. The image-detector an image-charge detector detects oscillations of the ions in the mass analyzer to generate a count or intensity of the ions.

The output of the detector is provided to a computing system 114 that may be external to, or incorporated into, the mass spectrometer. In general, the computing system 114 is in electronic communication with the detector 112 such that the computing elements are able to receive the signals generated from the detector 112. The computing system includes at least one processor and memory, both of which are hardware devices. The processor may include multiple processors (and/or processing cores) and may include any type of suitable processing components for processing the signals and generating the results discussed herein. Depending on the exact configuration, the memory (storing, among other things, mass analysis programs and instructions to perform the operations disclosed herein) can be volatile (such as RAM), non-volatile (such as ROM, flash memory, etc.), or some combination of the two. Other computing elements may also be included in the computing system 114. For instance, the computing system 114 may include storage devices (removable and/or non-removable) including, but not limited to, solid-state devices, magnetic or optical disks, or tape. The computing system 114 may also have input device(s) such as touch screens, keyboard, mouse, pen, voice input, etc., and/or output device(s) such as a display, speakers, printer, etc. One or more communication connections, such as local-area network (LAN), wide-area network (WAN), point-to-point, Bluetooth, RF, etc., may also be incorporated into the computing system 114.

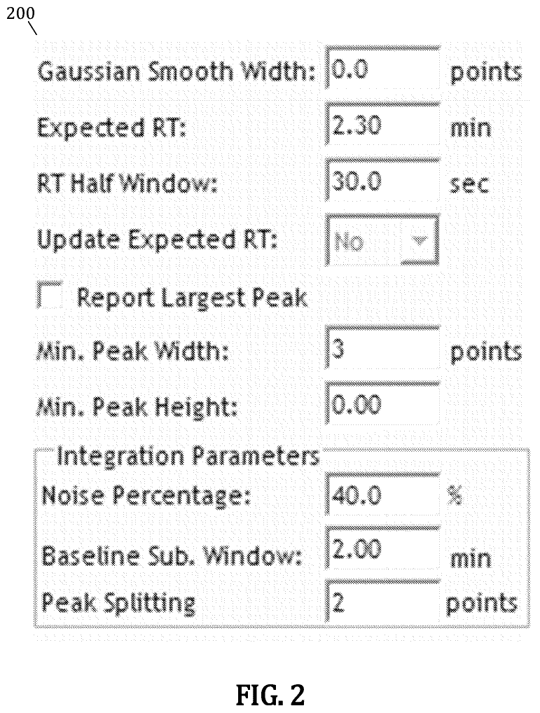

FIG. 2 is an example user interface 200 showing example peak-finding or peak integration parameters used by an example peak finding algorithm to integrate a peak. User interface 200 may be generated by one of the peak finding algorithms of SCIEX, for example. The user interface 200 allows a user to change the parameters to integrate or re-integrate a specific peak.

The peak integration parameters utilized in the present technology may include one or more of the parameters listed in the user interface 200. For instance, the present technology may determine one or more of the integration parameters that produce the best or most-accurate peak integration. The peak integration parameters may include parameters such as a smoothing parameter, an expected-time parameter, filtering parameters, a baseline parameter, and/or a peak-splitting parameter. The listed integration parameters in interface 200 are specific non-limiting examples of such integration parameters and include gaussian smooth width, expected retention time (RT), RT half-window, minimum peak width, minimum peak height, noise percentage, baseline subtraction window, and peak splitting. Even though only the bottom three are labeled as “integration parameters,” all the listed parameters may have an effect on the ultimate integration of the peaks in the ion data series.

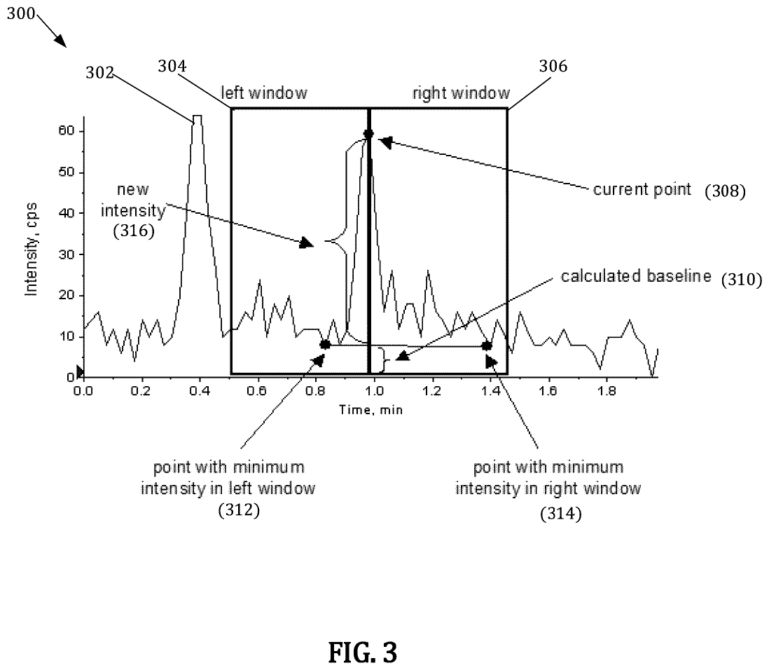

FIGS. 3-5 are provided below to facilitate discussion of an example peak finding and integration algorithm for an ion data series in a chromatogram, which uses the parameters of baseline subtraction window (in minutes), noise percentage and peak splitting factor. As should be appreciated, the example peak-finding algorithm discussed below is one example of a peak-finding algorithm, but other peak-finding algorithms are available that may utilize other parameters or parameter types.

FIG. 3 depicts an example chromatogram 300 explaining the use of the baseline subtraction window parameter. The chromatogram 300 includes an ion data series or ion signal 302. The first step in the peak integration algorithm is to apply a baseline subtraction filter to ion signal 302 in the chromatogram 300. This filter replaces each data point with its baseline subtracted value where the baseline 310 is determined as the line connecting the data point 312 on the left side of the current point 308 with minimum intensity (within the left baseline subtraction window 304) to the data point 314 on the right side with minimum intensity (within the right baseline subtraction window 306). The baseline subtraction window parameter determines the width of the windows 304, 306. The new intensity 316 is based on the remaining ion signal 302 above the baseline 310. Note that a different baseline 310 may be used is used for every data point in the ion signal 302.

The next step in the algorithm is to determine the noise level. The noise level is estimated by calculating the standard deviation of the smallest ‘noise percentage’ baseline subtracted data points. For example, the value of the noise percentage parameter may be 50%, the standard deviation of the half of the data points with lowest intensity is calculated (so if there are 100 points the least intense 50 are used). The peak-finding threshold is then set to the average of these data points plus twice their standard deviation.

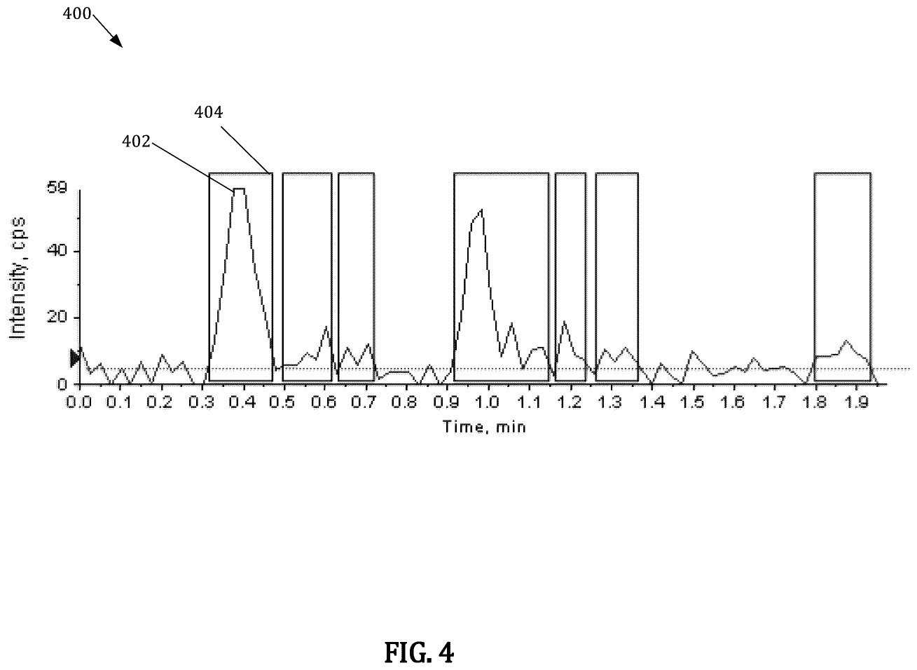

The next step of the algorithm is to identify peak ‘clusters.’ FIG. 4 depicts another chromatogram 400 with an ion signal 402 and cluster bounding boxes 404. The cluster bounding boxes 404 identify different peak clusters, and in the example chromatogram 400, seven peak clusters are identified. The starts of peak clusters are found by locating all places in the baseline subtracted data at which the intensity rises above the peak-finding threshold calculated above. The end of the cluster is the location at which the intensity falls below this threshold. In order for the cluster to be kept for further analysis, it must be the number of data points wide as set in the minimum peak width parameter.

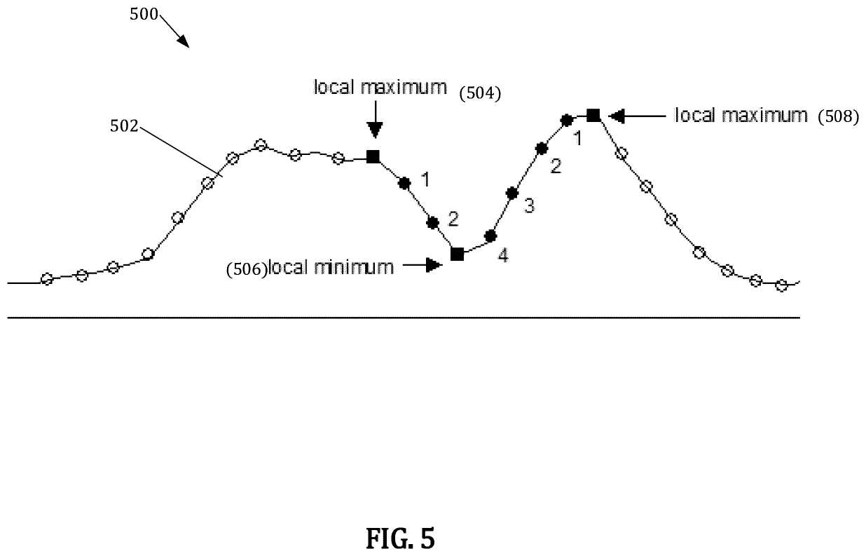

At this point in the algorithm, the analysis may revert to the raw data (i.e., before baseline subtraction). FIG. 5 depicts a portion of a chromatogram 500 with an ion signal 502. The various clusters are then divided into one or more separate peaks by dropping a vertical line from certain local minima 506 within the cluster to the baseline. The local minima 506 are those for which the number of consecutive rising points on each side exceeds or equals the specified value of the peak-splitting parameter. Setting this parameter large prevents clusters from being split into more than one peak. In chromatogram 500, two separate peaks will be found provided that the peak-splitting factor is two or less—only those points are counted which are strictly between the local maxima 504, 508 and the local minimum 506. Once the start and end of each peak are located, the peak area (i.e., the integrated area) and peak height are calculated.

Two other parameters may be considered at this point in the algorithm, including the minimum peak width (in terms of number of data points) and minimum peak height (in counts/second). If a peak is narrower than the peak minimum width or shorter than the minimum peak height, the peak is not reported.

The above peak-finding and integrating algorithm is just one example peak-finding algorithm, which may be referred to as the MQ4 algorithm. Other algorithms, such as the AutoPeak or SignalFinder algorithm from SCIEX of Framingham, Massachusetts, may also be used. Algorithms such as the AutoPeak algorithm differ from the algorithm above. For instance, in the AutoPeak algorithm, the algorithm is trained on results from a clean standard. In that training, the algorithm generates a function that describes the shape of the peak in the results mathematically. The function may be generated by fitting a combination of Gaussian curves that form a model of the peak. Then, when an ion signal for an unknown sample is received and analyzed, the AutoPeak algorithm takes the generated peak model and attempts to fit it to peaks in the new ion signal by stretching and/or scaling the peak model.

The AutoPeak algorithm and similar algorithms also utilize peak integration parameters to identify and integrate the peaks; although at least some of the integration parameters for the AutoPeak algorithm or similar algorithms may be different from the integration parameters for the MQ4 algorithm or similar algorithm. For instance, the peak integration parameters for the AutoPeak algorithm or similar algorithms may include smoothing parameters, filters, minimum peak height, minimum peak width, retention time parameters, and a sensitivity parameter (which helps decide when to split a peak into two peaks).

For the AutoPeak algorithm and similar algorithms, metrics are then generated indicated how well the peak model fits a peak in the new ion signal. These metrics may be referred to as peak quality metrics. The peak quality metrics may include a value indicating the difference between the identified peak and the peak model. For instance, a peak quality metric may be an average deviation from the identified peak and the peak model.

From the foregoing discussion, it should be appreciated how the different peak integration parameters may have an effect on how the peaks in the ion data series on the ultimate identification, definition, and integration of the peaks. For instance, changing the parameters may change how each peak is identified and defined, which similarly changes the area of the peak (i.e., the peak integration) and, ultimately, the determined ion amount and analyte concentration. Some peak integrations may be more accurate than others, and accordingly, some sets of peak-integration parameters are more accurate than others. The parameters, however, may need to change for each type of analysis or even for each type of peak that is present in the ion data series or ion signal. For instance, one set of integration parameters may be appropriate for one chromatogram but not for another chromatogram.

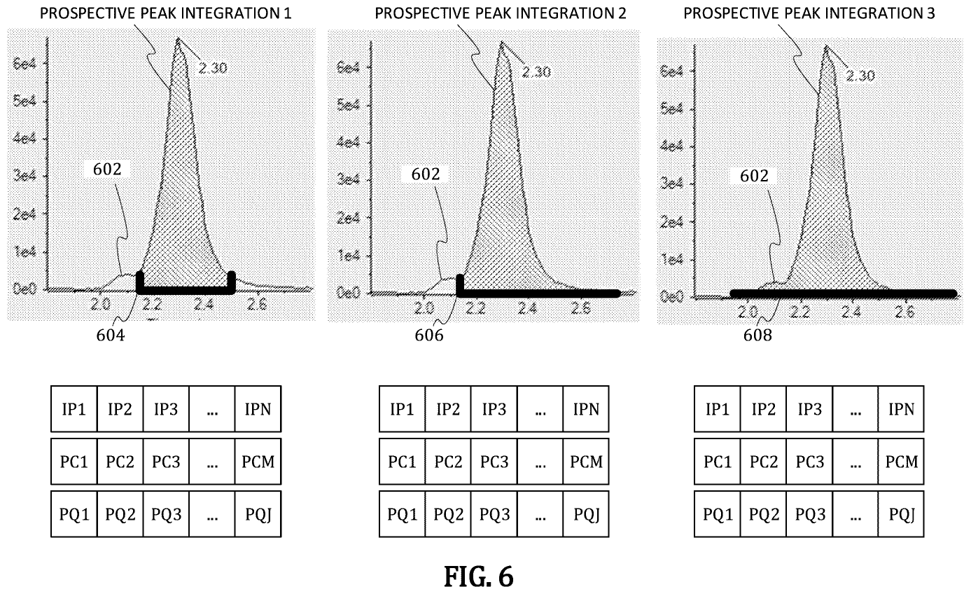

FIG. 6 depicts three example prospective peak integrations generated from three different sets of integrations parameters. The three prospective peak integrations include a first prospective peak integration (labeled prospective peak integration 1), a second prospective peak integration (labeled prospective peak integration 2), and a third prospective peak integration (labeled prospective peak integration 3). Each of the three prospective peak integrations are generated for the same ion data series or ion signal 602. While only three prospective peak integrations are depicted, it should be appreciated that dozens or hundreds of prospective peak integrations may be generated. In some examples, more than 50 or more than 100 prospective peak integrations may be generated.

Each of the prospective peak integrations is generated from a different set of integrations parameters, represented as an array of integration parameters IP1-IPN for N different integration parameters. As can be seen from the cross-hatched area and the peak baselines 604, 606, 608, the three prospective peak integrations are different from one another due to the difference in integration parameters. The peak baselines 604, 606, 608 may have vertical and horizontal components, represented by the thickened black lines. The vertical component of peak baselines 604, 606, 608 separate the area of peak from potentially interfering peaks. In some embodiments and for some algorithms, the peak baselines 604, 606, 608 may be curves.

As an example of the differences, a comparison of three examples prospective peak integrations shows that, for the second prospective peak integration, the peak baseline 606 excludes a small interfering peak 520 at the beginning of larger peak, but does not exclude the shoulder at the end of the larger peak. As a result, the second prospective peak integration may be considered a more accurate integration as compared to the third prospective peak integration which does not exclude the beginning or ending shoulder. In contrast, in the first prospective peak integration, peak baseline 604 excludes the shoulder at the end of peak in addition to the small interfering peak at the beginning of peak. Thus, the first prospective peak integration may be potentially considered more accurate than the second prospective peak integration.

Each of the prospective peak integrations may be characterized or defined by a set of peak characteristics, represented by the array of peak characteristic (PC) values PC1-PCM for M different peak characteristics. The peak characteristics may include integrated area of the peak, peak height, peak start time, peak end time, center time, peak width, and peak smoothness, among other types of peak characteristics. The peak characteristics may be measured using a variety of different manners as long as the measurement technique is used consistently for all the prospective peak integrations. For instance, peak width may be measured using a full width at half maximum (FWHM) technique. That technique, or any other technique, may be used as long as it is consistently used for generated the equivalent peak characteristic for all the prospective peak integrations. For instance, peak width may be measured at other percentages of peak height and ratios of peak widths may also be utilized. Because each of prospective peak integrations are different, the set of peak characteristics for each of the prospective peak integrations may also be different. However, one or more peak characteristics may be shared amongst different prospective peak integrations. For example, two different prospective peak integrations generated from different integration settings may, in fact, generate the same integrated area.

Each of the prospective peak integrations may also be characterized by peak quality metrics. The peak quality metrics may be those discussed above, such as an average deviation from the identified peak and the peak model when using a peak-finding algorithm similar to the AutoPeak algorithm. The peak quality metrics may also be presented as an array of peak quality metric (PQ) values PQ1-PQJ for J different peak quality metrics.

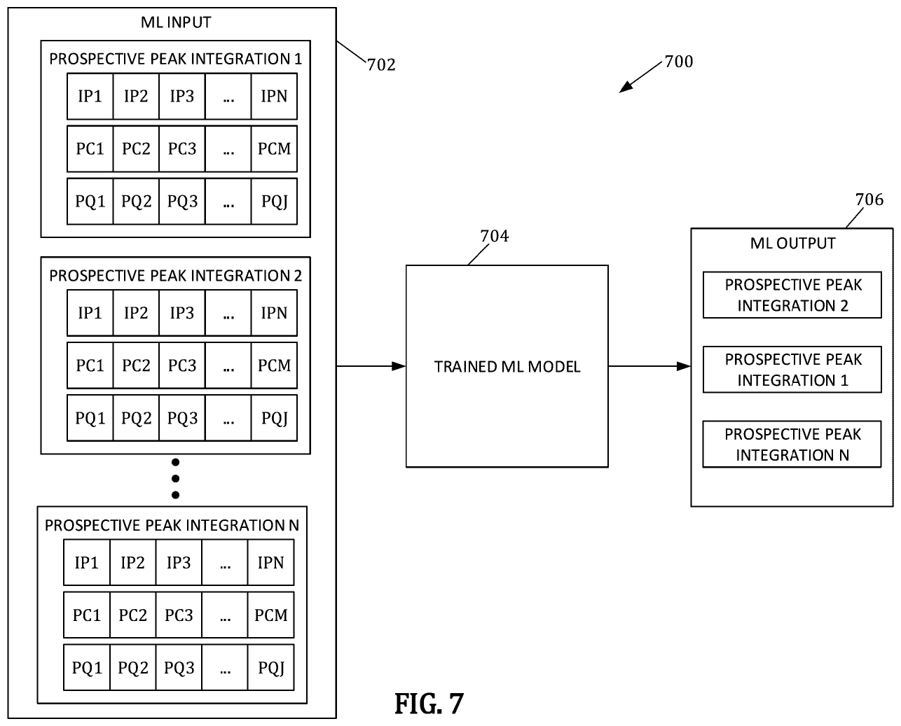

FIG. 7 depicts an example system 700 for predicting top prospective peak integrations using a trained machine learning (ML) model 704. The input 702 for the ML model 704 may be generated based on the prospective peak integrations. The input 702 may include different features for each of the generated prospective peak integrations. For example, the input 702 may include one or more input parameters, peak characteristics, and/or peak quality metrics for each of the generated prospective peak integrations. In some examples, the input 702 may include only one of the peak characteristics, such as the peak integration area, for each of the prospective peak integrations. The integration parameters, peak characteristics, and/or peak quality metrics are correlated with the corresponding prospective peak integration such that the ML model 704 is able to predict or generate the top prospective peak integrations based on the input 702. In some examples, the input 702 may further include the ion data series or ion signal for which the prospective peak integrations were generated.

The generated input 702 is then provided to the trained ML model 704. The ML model may be a neural network or other suitable ML model, such as a support vector machine, a K-nearest neighbors algorithm, a hidden Markov model, or a random forest. The ML model processes the input to generate an output 706. The output 706 includes a ranking (e.g., scoring) of the prospective peak integrations included in the input 702. For instance, in some examples the ranking is represented by a score assigned each of the prospective peak integrations, even where the prospective peak integrations are not explicitly sorted by that score. In some examples, the output 706 includes a single prospective peak integration that is the top ranked (e.g., highest or best score) prospective peak integration. Based on the output 706, one or more top prospective peak integrations may be identified. For instance, the top 3 or 5 ranked prospective peak integrations in the output 706 may be selected.

The ML model may be trained using a supervised training technique. The training data may be generated from prior-manual selections identifying the best prospective peak integrations for prior data. For instance, data regarding which prospective peak integrations or integration parameters have been previously selected by users may be used as a ground truth for the ML model during training. Alternatively or additionally, synthetic data may also be generated and used for training of the ML model. For example, synthetic data may be generated that simulates known results or concentrations. Noise or other complicating factors may be introduced into the synthetic data to create modified data. Prospective peak integrations may then be generated for the modified data. The best or most accurate prospective peak integration may be the prospective peak integration that matches the known concentration. Thus, that most accurate prospective peak integration may be used as the ground truth for the set of prospective peak integrations when training the ML model.

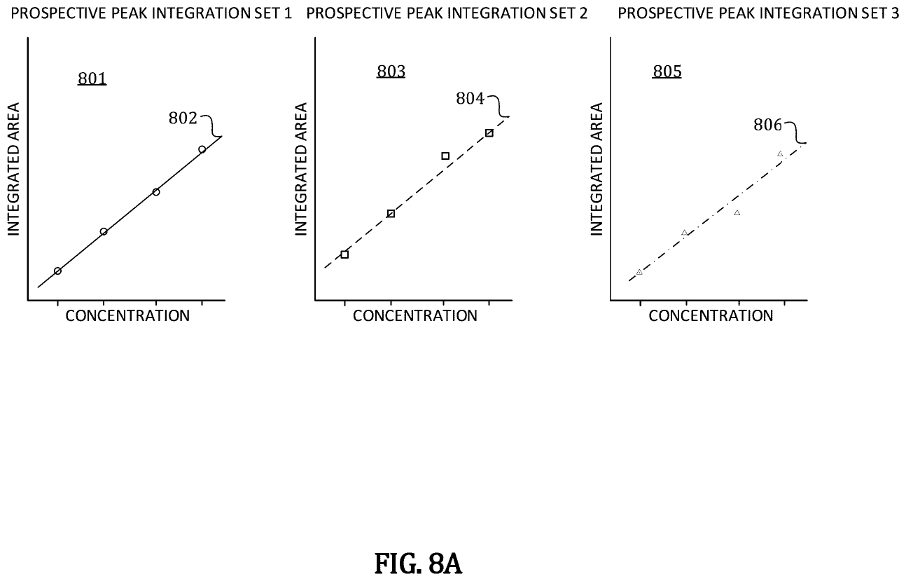

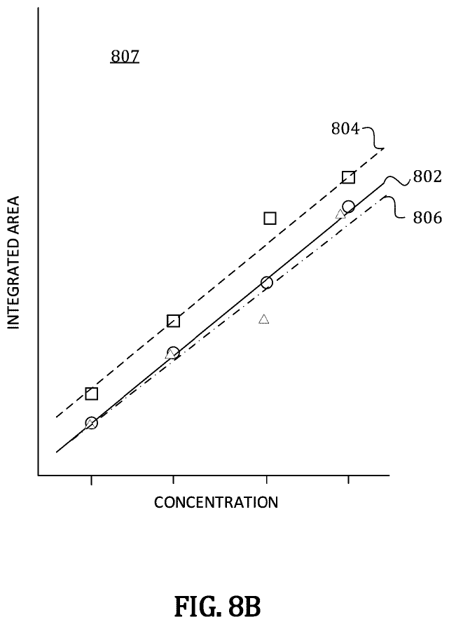

FIG. 8A depicts example plots 801, 803, 805 for standardized results using different prospective peak integrations. The example plots 801, 803, and 805 each include concentration of the tested sample on the x-axis and integrated area on the y-axis. In this example, four samples were analyzed each having known concentrations of an analyte. The analyte concentrations were linearly increasing. For instance, the second concentration was twice the first concentration, the third concentration was three times the first concentration, and the fourth concentration was four times the first concentration.

For each prospective peak integration set, the integration parameters of the prospective peak integration are applied to ion data series for the samples with known analyte concentrations, and the resultant integration area is plotted on each respective plot. For example, for first prospective peak integration set, the integration areas according to the integration parameters for the first prospective peak integration set are plotted as circles in plot 801. For the second prospective peak integration set, the integration areas according to the integration parameters for the second prospective peak integration set are plotted as squares in plot 803. For the third prospective peak integration set, the integration areas according to the integration parameters for the third prospective peak integration set are plotted as triangles in plot 805.

For each of the prospective peak integration plots, a line or curve may be fitted to the plotted integrated areas. For instance, for the first prospective peak integration set a first fitted curve or line 802 is generated. For the second prospective peak integration set, a second fitted curve or line 804 is generated. For the third prospective peak integration set, a third fitted curve or line 806 is generated. For each of the fitted lines 802, 804, 806, a regression or curve fit value may be determined that describes or represents the accuracy of the fitted line to the data. The curve fit value may be a coefficient of determination (R²) or other similar value.

Because the samples have known concentrations that increase linearly, perfect integrated areas would form a straight line (e.g., have a coefficient of determination of 1). Accordingly, the prospective peak integrations may be ranked based on their regression values. For instance, the prospective peak integrations that have the highest (or best) curve fit values may be ranked highest (or best) for use in generating predicted integrations.

FIG. 8B depicts the plots 801, 803, 805 of FIG. 8A combined into a single plot 807. By combining the plots 801, 803, 805 for each prospective peak integration together, additional metrics may be generated that indicate the accuracy of the integrated areas of each prospective peak integration. When plotted together, each of the fitted lines 802, 804, 806 may be further assessed and scored. As an example, the deviation of the integrated areas (represented by the circle, triangle, and square respectively) from the respective fitted line may be evaluated. For instance, for the second fitted line 804 generated from the second prospective peak integration, the standard deviation of the integrated areas across all prospective peak integrations for the first concentration is greater than that of the first fitted line 802 or the third fitted line 806. A fitted line having a large deviation (e.g., standard deviation) may be indicative that the corresponding prospective peak integration is less suitable for use. Accordingly, the prospective peak integrations may be ranked based on their deviations. Prospective peak integrations with the smallest deviations may be ranked higher than those with the smallest deviations.

To account for both the curve fit value and the deviation value for each prospective peak integration, an accuracy score may be generated that is based on the curve fit value and the deviation value, among potential other values that may represent the accuracy of the prospective peak integration. Each of values going into the score may weighted. For instance, the accuracy score may be represented by the following equation:

$$[ Accuracy Score ] = W 1 [ CurveFit ] + W 2 [ Deviation ] ,$$

where W1 is a weight for the curve fit value and W2 is the weight for the deviation value. Of note, the deviation value may be represented as the reciprocal (e.g., 1/Deviation) such that a smaller deviation leads to a higher accuracy score. Prospective peak integrations with higher (or better) accuracy scores may be ranked more highly.

Based on the ranking of the prospective peak integrations based on curve fit, deviation value, and/or accuracy score, a subset of the highest ranked prospective peak integrations may be selected. For example, the top 50% or top third of the prospective peak integrations may be selected. The top ranked prospective peak integrations may then be used in different manners for determining ion amounts, and ultimately concentrations, for unknown samples. For example, the subset of selected prospective peak integrations may then be used as input for the machine learning model discussed above with reference to FIG. 7. In other examples, the top-ranked prospective peak integrations (e.g., top 2-5 prospective peak integrations) may be presented to the user for selection for use with the unknown sample. In another example, the top-ranked prospective peak integration may be used for integrating the peaks of an unknown sample (e.g., integrating peaks of a chromatogram for an unknown sample). Additional rules may also, or alternatively, be applied for further narrowing down the top-ranked prospective peak integrations. For instance, the top-ranked prospective peak integrations may be further limited to prospective peak integrations having peak characteristics and/or peak quality metrics within a certain range. The prospective peak integrations may also be limited to prospective peak integrations having integration parameters within a certain range.

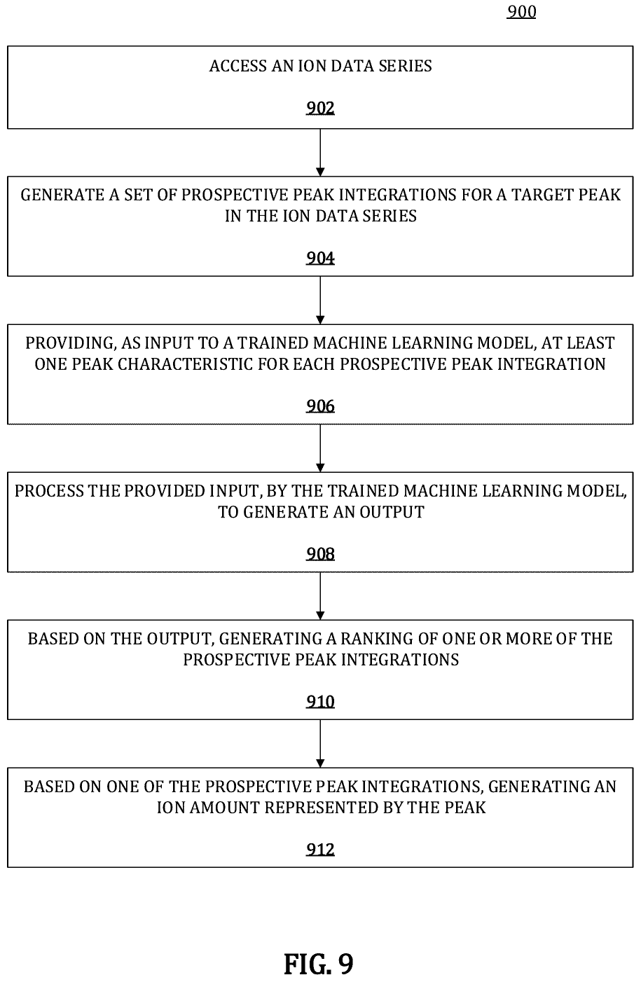

FIG. 9 depicts an example method 900 for improving mass spectrometry measurements. The operations of method 900 may be performed by systems discussed herein, such as system 100, or components thereof. For example, the operations of method 900 may be performed by one or more processors in the system according to instructions stored in memory of the system. At operation 902, an ion data series (e.g., an ion data signal) is accessed. The ion data series is for an ion count rate generated from ions detected by a detector of a mass spectrometry system. In some examples, the ion data series may be a chromatogram or part of a chromatogram. Each data point within the ion data series may be indicate an ion count rate and sampling interval time. For example, each sampling interval of the detector may generate an ion-count-rate data point.

At operation 904, a set of prospective peak integrations for a target peak in the ion data series is generated. Each prospective peak integration in the set of prospective peak integrations is generated based on a different set of peak integration parameters. Each prospective peak integration is also characterized by at least one peak characteristic. In some examples, each of the prospective peak integrations may also be characterized by one or more peak quality metrics.

At operation 906, at least one peak characteristic for each prospective peak integration is provided as input into a trained machine learning model. More than one peak characteristic for each prospective peak integration may be provided as input to the trained machine learning model. In some examples, one or more of the peak integration parameters for each prospective peak integration may also be provided as the input. Where available, one or more peak quality metrics for each prospective peak integration may also be provided as the input. The ion data series, or a portion thereof, may also be provided as the input.

At operation 908, the trained machine learning model processes the input provided at operation 906. The machine learning model then generates an output, which may be a ranking or an indication of the ranking of the prospective peak integrations used for the input. At operation 910, based on the output of machine learning model, a ranking/scoring of one or more of the prospective peak integrations is generated. The ranking indicates the likely suitability and/or accuracy of the prospective peak integration, as discussed above.

At operation 912, based on one of the prospective peak integrations, an ion amount (e.g., the integrated area) for the target peak is generated. The ion amount for the target peak may represent a concentration of an analyte for the sample. Accordingly, operation 912 may also include generating a concentration of the analyte based on one of the prospective peak integrations.

Operation 912 may also include additional sub-operations to determine which prospective peak integration to use to generate the ion amount. For example, one or more of the prospective peak integrations may be displayed based on the ranking of the prospective peak integrations. For instance, the top-ranked two, three, five, etc. prospective peak integrations may be displayed. A processor may cause such a display by sending a display signal to a monitor or via a data transmission to a display device. A selection of one of the displayed prospective peak integrations may then be received, such as via user selection using an input device or touch input. The generated ion amount may then be based on the selected prospective peak integration.

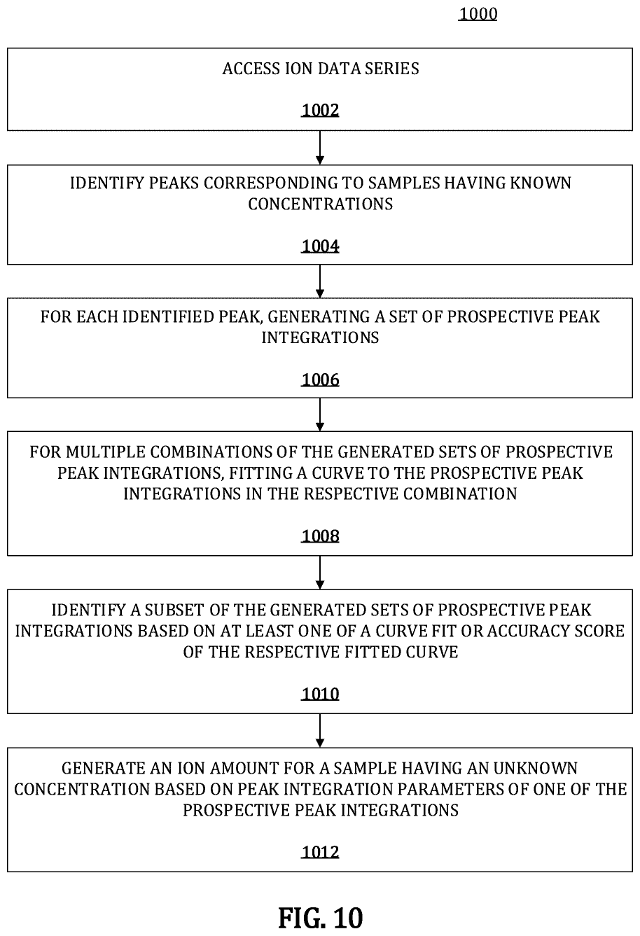

FIG. 10 depicts another example method 1000 for improving mass spectrometry measurements. Similar to method 900 in FIG. 9, the operations of method 1000 may be performed by systems discussed herein, such as system 100, or components thereof. For example, the operations of method 1000 may be performed by one or more processors in the system according to instructions stored in memory of the system. At operation 1002, one or more ion data series (e.g., ion data signals) are accessed. The ion data series is for an ion count rate generated from ions detected by a detector of a mass spectrometry system. In some examples, the ion data series may be a chromatogram or part of a chromatogram. Each data point within the ion data series may be indicate an ion count rate and sampling interval time. For example, each sampling interval of the detector may generate an ion-count-rate data point. The ion data series that are accessed in operation 1002 are for samples having known concentrations of an analyte.

At operation 1004, peaks corresponding to analytes having the known analyte concentration in the sample are identified. The identified peaks may be identified in different ion data signals or in a single continuous ion data signal depending on how the experiment is executed. At operation 1006, for each identified peak, a set of prospective peak integrations is generated. Each of the prospective peak integrations is generated according to different peak integration parameters. For example, for the first peak corresponding to the first known analyte concentration, a first set of prospective peak integrations are generated. A second set of prospective peak integrations are also generated for the second peak corresponding to the second known analyte concentration. The first and second sets of prospective peak integrations are generated based on the same set of integration parameters. For instance, a first prospective peak integration is generated for the first peak and the second peak according to the same integration parameters. A second prospective peak integration is also generated for the first peak and the second peak according to the integration parameters that are different from the integration parameters used to generate the first prospective peak integration.

At operation 1008, for multiple combinations of the generated sets of prospective peak integrations, a line or curve is fitted to the prospective peak integrations in the respective combination. For example, the integrated areas of for each of the prospective peak integrations generated for the known concentrations may be correlated with and/or plotted against the known concentrations, such as in FIG. 8A described above. For instance, the integrated areas according to the prospective peak integrations may be stored in a manner that correlates the integrated area to the corresponding concentration, such as an array of ordered pairs. A curve may then be fitted to integrated areas for each prospective peak integration. The curve may be a line.

At operation 1010, a subset of the generated prospective peak integrations is identified based on a at least one of a curve fit or accuracy score of the respective fitted curve for the corresponding prospective peak integration. The curve fit may be a regression value such as the coefficient of determination (R²) or other similar value. The accuracy score for the fitted line may be based on the deviation and/or the curve fit value, as discussed above with respect to FIG. 8B. The curve fit and/or accuracy score may thus be used to rank the prospective peak integrations. The subset of prospective peak integrations identified in operation 1010 may then be the top-ranked prospective peak integrations that are above some threshold. For example, the top half or top third of the prospective peak integrations may be selected as the subset of prospective peak integrations. In other examples, the top one, two, three, four, or five prospective peak integrations may be selected as the subset.

At operation 1012, an ion amount (e.g., integrated area) for a sample having an unknown analyte concentration is generated based on the peak integration parameters of one the prospective peak integrations in the subset of prospective peak integrations identified in operation 1010. For example, the integration parameters for the prospective peak integration having the best curve fit and/or accuracy score may be used. In other examples, the subset of prospective peak integrations may be displayed to a user for selection.

In another example, the subset of prospective peak integrations identified in operation 1010 may be used as input for a trained machine learning model, such as discussed above with respect to method 900 in FIG. 9. For example, each of the subsets of prospective peak integrations may be characterized by one or more peak characteristics, and those one or more peak characteristics may be used as the input to the trained machine learning model. Other types of input based on the prospective peak integrations may also be utilized. The output of the machine learning model may then be used to rank the prospective peak integrations and ultimately select a prospective peak integration for which the integration parameters are to be used for determining an ion amount for the sample with the unknown analyte concentration.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved. Further, as used herein and in the claims, the phrase “at least one of element A, element B, or element C” is intended to convey any of: element A, element B, element C, elements A and B, elements A and C, elements B and C, and elements A, B, and C.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method for improving mass spectrometry system measurement, the method comprising:
accessing an ion data series for an ion count rate generated from ions detected by a detector of a mass spectrometry system;
generating a set of prospective peak integrations for a target peak in the ion data series, wherein each prospective peak integration in the set of prospective peak integrations is generated based on a different set of peak integration parameters, and each prospective peak integration is characterized by at least one peak characteristic;
each prospective peak integration in the set of prospective peak integrations;
processing the provided input, by the trained machine learning model, to generate an output from the trained machine learning model;
based on the output, generating a ranking of one or more of the prospective peak integrations; and
based on one of the prospective peak integrations, generating an ion amount represented by the target peak.

2. The method of claim 1, further comprising:
causing a display of one or more of the prospective peak integrations based on the ranking;
receiving a selection of one of the displayed prospective peak integrations; and
wherein generating the ion amount is based on the selected prospective peak integration.

3. The method of claim 1, wherein the peak integration parameters include at least one of a smoothing parameter, an expected-time parameter, a filtering parameter, a baseline parameter, or a peak-splitting parameter.

4. The method of claim 1, wherein the at least peak characteristic includes at least one of: an integrated area, peak height, peak start time, peak end time, center time, peak width, and peak smoothness.

5. The method of claim 1, wherein each prospective peak integration in the set of prospective peak integrations includes at least one respective peak quality metric, and the respective peak quality metrics are also included as input into the trained machine learning model.

6. The method of claim 1, wherein one or more of the peak integration parameters are also included as input to the trained machine learning model.

7. The method of claim 1, wherein the set of prospective peak integrations includes at least 50 prospective peak integrations.

8. The method of claim 1, wherein the trained machine learning model is one a neural network, a support vector machine, a K-nearest neighbors algorithm, a hidden Markov model, or a random forest.

9. The method of claim 1, wherein the ion data series is part of a chromatogram.

10. The method of claim 1, wherein data points within the data series indicate an ion count rate and sampling interval time.

11-15. (canceled)

16. A method for improving mass spectrometry system measurement, the method comprising:
accessing an ion data series for an ion count rate generated from ions detected by a detector of a mass spectrometry system;
generating, according to first peak integration parameters, a first prospective peak integration for an identified peak in the ion data series, wherein the first prospective peak integration is characterized by first peak characteristics;
generating, according to second peak integration parameters, a second prospective peak integration for the identified peak in the ion data series, wherein the second prospective peak integration is characterized by second peak characteristics;
providing, as input to a trained machine learning model:
the first peak characteristics; and
the second peak characteristics;
processing the provided input, by the trained machine learning model, to generate an output from the trained machine learning model;
based on the output, generating a ranking of the first prospective peak integration and second prospective peak integration; and
based on at least one of the first prospective peak integration or the second prospective peak integration, generating an ion amount represented by the peak.

17. The method of claim 16, further comprising:
causing the display of at least one of the first prospective peak integration or the second prospective peak integration based on the ranking;
receiving a selection of one of the first prospective peak integration or the second prospective peak integration; and
wherein generating the ion amount is based on the selected prospective peak integration.

18. The method of claim 16, wherein the peak integration parameters include at least one of a smoothing parameter, an expected-time parameter, a filtering parameter, a baseline parameter, or a peak-splitting parameter.

19. The method of claim 16, wherein the peak characteristics include at least two of: an integrated area, peak height, peak start time, peak end time, center time, peak width, and peak smoothness.

20. The method of claim 16, wherein the trained machine learning model is one a neural network, a support vector machine, a K-nearest neighbors algorithm, a hidden Markov model, or a random forest.

21. The method of claim 16, wherein the first prospective peak integration has a first peak quality metric, the second prospective peak integration has a second peak quality metric, and the input to the trained machine learning model further includes the first peak quality metric and the second peak quality metric.

22. A method for improving mass spectrometry system measurement, the method comprising:
accessing an ion data series for an ion count rate generated from ions detected by a detector of a mass spectrometry system;
identifying peaks corresponding to samples having known analyte concentrations;
for each identified peak, generating a set of prospective peak integrations for the identified peak in the ion data series, wherein each of the prospective peak integrations is generated according to different peak integration parameters;
for multiple combinations of the generated sets of prospective peak integrations, fitting a curve to the prospective peak integrations in the respective combination;
identifying a subset of the generated prospective peak integrations based on at least one of a curve fit or accuracy score of the respective fitted curve; and
generating an ion amount for a sample having an unknown concentration based on peak integration parameters of one of the prospective peak integrations in the identified subset of the prospective peak integrations.

23. The method of claim 22, wherein each prospective peak integration is characterized by peak characteristics.

24. The method of claim 22, further comprising:
providing, as input to a trained machine learning model, the peak characteristics for the subset of prospective peak integrations;
processing the provided input, by the trained machine learning model, to generate an output from the trained machine learning model; and
based on the output, generating a ranking of one or more of the prospective peak integrations in the subset of prospective peak integrations.

25. The method of claim 22, further comprising:
causing a display of one or more of the prospective peak integrations in the subset of prospective peak integrations based on the ranking;
receiving a selection of one of the displayed prospective peak integrations; and
wherein generating the ion amount is based on the selected prospective peak integration.
